(19) United States
(12) Patent Application Publication
(10) Pub. No.: US 2023/0068366 A1
(43) Pub. Date: Mar. 2, 2023

(54) SYSTEM FOR AIDING PLACEMENT OF A NASOGASTRIC TUBE OR A NASODUODENAL TUBE AND METHOD FOR USING THE SAME

(71) Applicants: MediVisionTech Co., Ltd., Tainan City (TW); National Cheng Kung University Hospital, Tainan City (TW)

(72) Inventors: Chien-Cheng CHEN, Tainan City (TW); Chiao-Hsiung CHUANG, Tainan City (TW)

(21) Appl. No.: 17/894,389

(22) Filed: Aug. 24, 2022

Related U.S. Application Data
(60) Provisional application No. 63/237,142, filed on Aug. 26, 2021.
(60) Provisional application No. 63/285,070, filed on Dec. 1, 2021.

(51) Int. Cl. A61J 15/00; A61B 1/00; A61B 1/05

(52) U.S. Cl. CPC A61J 15/0003; A61B 1/00052; A61B 1/00154; A61B 1/05; A61B 1/233

(57) ABSTRACT

A system for aiding placement of a nasogastric tube or a nasoduodenal tube and a method for using the same is disclosed, and the system comprises that of: a placing tube; a three-way connector, having a first port, a second port, and a third port, the first port being on the opposite end to the second port, the first port being adjacent to the third port, the first port being connected with a proximal end of the placing tube, the third port being adapted for pumping air or fluid into the three-way connector; an endoscope, having an insertion tube and an image-capturing device, the distal end of the insertion tube being a bending section, the image-capturing device being located in the front end of the bending section; and a display unit, connected with the endoscope.

### TECHNICAL FIELD

The present invention is a system for aiding placement of a nasogastric tube or a nasoduodenal tube and method for using the same, more particularly a system for helping to place nasogastric/nasoenteric tubes and a method thereof.

### BACKGROUND

Providing nutrition via nasogastric tubes is very important for patients with some specific diseases, e.g. abnormal narrowing of the esophageal lumen, dysphagia caused by a stroke, or low consciousness caused by serious health conditions, etc. According to literatures published by Taiwan's Ministry of Health and Welfare, there were around 200,000 inpatients (˜10% of all inpatients) who required nasogastric intubation in 2019.

Although nasogastric tube feeding is commonly performed in medical settings, there are two disadvantages as follows. The first disadvantage is that nasogastric tubes are mainly blindly inserted. After the nasogastric tube is inserted into the nasal cavity, the insertion relies very much on the healthcare workers' experience and techniques. This intubation method may require repeated insertion to confirm that the nasogastric tube has been placed into the correct tract. Such repeated insertion would cause great discomfort for the patients. The other disadvantage is that it might be difficult to identify whether the nasogastric tube has been correctly placed to the right position. Common types of malposition include placing the tube external to the gastrointestinal tract (e.g. tracheal malposition, esophageal perforation, or pleural perforation) and “improper placement of the nasogastric tube” (e.g. not reaching the correct tract, U-turn, and entanglement whirling). This kind of malposition could be seen in patients critically ill, with coma or near coma, or with abnormal swallowing and coughing functions.

The international standard methods for confirming the position of the nasogastric tube mainly are that of: (1) X-ray assisted placement method, which is Gold standard but time-consuming with associated expense (50-500 US dollars for taking X-ray in the United States), and (2) Trying to Pump back the gastric juice from the tube and checking the pH value to confirm the position of the nasogastric tube.

On the other hand, the methods above have the following disadvantages. The first disadvantage is that the X-ray machine is not always immediately available, and the other is that aspiration of gastric juices cannot be collected from some patients for confirmation of the position of the nasogastric tube. Clinically, malpositioning therefore still occurs from time to time and cause aspiration pneumonia, serious harm, a vegetative state, or even death.

### SUMMARY

The main objective of the present invention provides a system for aiding placement of a nasogastric tube or a nasoduodenal tube. In this invention, the placing tube is connected to a three-way connector, which provides a channel that allows an endoscope to enter the lumen of the placing tube. Then the medical staff can perform the intubation of the nasogastric/nasoduodenal tube with the real-time images provided by the endoscope. In this way, the present invention highly decreases the error rate of intubation and improves the safety.

The other objective of the present invention provides a method for aiding placement of a nasogastric tube or a nasoduodenal tube. The method is to insert the endoscope into the placing tube via the three-way connector. Then the placing tube is placed into human body. If needed, air flow can be applied to inflate a cavity of the body lumen via a port of the three-way connector. Thus, the endoscope can capture better and clearer images to help putting the placing tube into the target position. The method speeds up the placement procedure as well as prevents misplacement.

To achieve the objectives described above, an embodiment of the present invention discloses a system for aiding placement of a nasogastric/nasoduodenal tube as follows. The system comprises: a placing tube; a three-way connector, an endoscope, and a display unit. The nasogastric/nasoduodenal tube is a regular nasogastric/nasoduodenal tube which can be found in the market. The three-way connector consists of a first port, a second port, and a third port. The first port is on the opposite end of the second port while it is adjacent to the third port. The first port is for connecting to the placing tube, and the third port is designed to pump the air or fluid into the placing tube. The endoscope is a device with an insertion tube and an image-capturing device. There is a bending section with an image-capturing device at the distal end of the insertion tube for changing the distal direction of the endoscope. Finally, a display unit is connected to the endoscope for demonstrating and recording real-time images.

The endoscope can be inserted into the second port of the three-way connector and then enter the placing tube from the first port of the three-way connector. The bending section of the endoscope can go throughout from the distal end of the placing tube to a cavity of the human body. Then the air or the fluid can be pumped into the third port of the three-way connector to inflate the cavity of the human body. As a result, the image-capturing device located at the distal end of the endoscope can capture better real-time images in the cavity of the human body. The real-time images are then shown on the display unit. By watching the real-time images of the cavity, the placing tube manipulated by the endoscope can be guided to enter the target lumen. After the confirmation of location of the placing tube by seeing the real-time images, the endoscope will be pulled back from the second port of the three-way connector. And then the three-way connector is removed. The intubation procedure is finished.

Preferably, the system comprises a cap with a hole structure that the endoscope can be combined with so that the cap can fit to the second port of the three-way connector when the endoscope goes through the three-way connector to prevent air leakage from the second port as well as fix the relative position of the endoscope and the three-way connector.

second port Preferably, the system further comprises a direction controller for the endoscope so that the direction of the distal end of the endoscope can be controlled to guide the placing tube into the target lumen.

Preferably, the endoscope has a light source, emitting a light to the lumen of the human body for better images capturing.

Preferably, the display unit shows real-time images of the lumen of the human body captured by the image-capturing device.

Preferably, the bending part comprises a first body, a second body, a first pivot element, and a second pivot element: The first body has eight concave shapes located in the four directions on the top and the bottom surfaces of the first body. There is a hole located in the middle of each of the concave shape penetrating through the first body. Similar to the first body, the second body also has eight concave shapes and four holes in the same location as the first body does. The first pivot element has convex shapes on the top and bottom surfaces and a hole in the middle of the convex shapes penetrating through the first pivot element. Similar to the first pivot element, the second pivot element also has convex shapes at two surfaces with a hole in the same location as the first pivot element does. The bending section is assembled as follows: The convex shapes of first and second pivot elements fit to one concave shape and that in the opposite direction of one surface of the first body; The second body is then put to the other surface of the two pivot elements with the concave shapes of the second body fitting to the convex shapes of the pivot elements. A first wire is applied through the holes in the corresponding location to connect the first body, the first pivot element, and the second body while a second wire is applied through the holes in the corresponding location to connect the first body, the first pivot element, and the second body.

Preferably, the location of the holes of the concave shapes of the first body without connecting to the pivot element axially corresponds to the location of the holes of the concave shapes of the second body.

Preferably, the bending part has a third wire and a fourth wire. The third wire is axially going from a hole without connecting to the pivot element of the first body to the corresponding hole of the second body while the fourth wire is axially going from the other hole without connecting to the pivot element of the first body to the corresponding hole of the second body. As a result, the axis of the bending section will move towards the third wire when pulling the third wire while axis of the bending section moves towards the fourth wire when pulling the fourth wire.

Preferably, the bending part further comprises a third body, a third pivot element and a fourth pivot element. The mechanical structure of the third body is the same as the first/second bodies while the mechanical structure of the third/fourth pivot elements is the same as the first/second pivot elements. The third/fourth pivot elements can be connected to the second body, similar to the way of the first/second pivot elements connecting to the first body. The third body, then, is assembled to the bending section by fitting two concave shapes of the third body to the third/fourth pivot elements, like the way the second body does. As a result, the third body, the third pivot element and the fourth pivot element are connected to the bending section.

Preferably, the first wire passes through the corresponding holes in the first body, the first pivot element, the second body, and the third body, respectively, while the second wire passes through the corresponding holes in the first body, the second pivot element, the second body, and third body, respectively. As a result, the axis of the bending section will move towards the first wire when pulling the first wire while axis of the bending section moves towards the second wire when pulling the second wire.

Preferably, the third wire passes through the corresponding holes in the first body, the second body, the third pivot element, and the third body, respectively, while the fourth wire passes through the corresponding holes in the first body, the second body, the fourth pivot element, and the third body, respectively. As a result, the axis of the bending section will move towards the third wire when pulling the third wire while the axis of the bending section moves towards the fourth wire when pulling the fourth wire.

Preferably, the bending section comprises a first body and a second body. The first body has four concave shapes located in the four directions on the bottom surface and two convex shapes in the two directions on the top surfaces. The position of one of two convex shapes is located in a position of the bottom surface being corresponding to the position where one concave shape is located on the top surface while the other convex shape is located in the position being opposite to the position of the first convex shape. Two holes penetrating through the first body are located in the positions corresponding to the two concave shapes on the top surface but the shape there is flat at the bottom surface. Two holes penetrating through the first body are located in the positions corresponding to the two concave shapes on the top surface but the shape there is flat at the bottom surface. The mechanical structure of the second body is the same as the first body. The first body and the second body are assembled by performing two steps. First, fit the convex shapes of the second body to the concave shapes of the first body by aligning the position of holes of the two bodies. Then, two wires are applied to pass through two holes of the first body and the corresponding holes of the second body. As a result, when pulling one the of two wires, the axis of the bending section will move towards the position of the pulled wire.

Preferably, two holes penetrating the first/second bodies are added to the middle of the other two convex shapes at the top surface, meaning the two holes are located in the middle of the two concave shapes at the bottom surface.

Preferably, the bending section comprises another two wires, which are passing through the holes the middle of the two convex shapes at the bottom surface of the first/second bodies, meaning the two wires are also passing through two concave shapes at the top surface of the first/second bodies.

Preferably, the bending section comprises a first body and a second body. The first body has a convex shape on the top surface, a concave shape on the bottom surface, and two holes passing from the top surface to the bottom surface. The position of the convex shape on the top surface is corresponding to the concave shape at the bottom surface. There are two planes on the two sides of the convex shape on the top surface. One hole is located one plane passing from the top surface to the bottom surface. The other hole is located on the other plane passing from the top surface to the bottom surface. The mechanical structure of the second body is the same as the first body. The first body and the second body are assembled together by fitting the convex shapes of the second body to the concave shapes of the first body with the holes on one body aligned with those on the other. Then, two wires are applied to pass through two holes of the first body and the corresponding holes of the second body. As a result, when pulling one of the two wires, the axis of the bending section will move towards the wire being pulled.

To achieve the second objective above, an embodiment of the present invention discloses a method for aiding placement of a nasogastric tube or a nasoduodenal tube, and it comprises the following steps: insert the insertion part of an endoscope from the second port of the three-way connector and through the proximal end of the placing tube via the first port of the three-way connector until the bending section of the endoscope sticks out of the distal end of the placing tube; put the placing tube into the natural tract of a human body; inject air or fluid to the third port of the three-way connector in order to inflate a cavity of the human body if needed; determine the position of the placing tube by viewing the images captured by the image-capturing device located in the distal end of the endoscope and shown on the display unit instantaneously.

Preferably, for the step of inserting the insertion part of an endoscope into the second port of a three-way connector, then going into the proximal end of a placing tube via the first port of the three-way connector, the endoscope can be combined with a cover through a hole structure of the cover and then the cover fits to the second port of the three-way connector so that the cover can prevent leakage of air or fluid from the second port.

Preferably, for the step of an image-capturing device of the endoscope capturing the images, which are shown on the display unit instantaneously, the placing tube is inserted to the target position by viewing the real-time captured images while using a direction controller to control the distal end of the endoscope to place the placing tube to the target position.

Preferably, for the step of putting the placing tube into a natural tract of the human body, a light source emits a light into the natural tract of the human body.

Preferably, for the step of the light source emitting the light into a natural tract of the human body, the images captured by the image-capturing device contain information of internal organs/tissues reflected by the light. The images of internal organs/tissues are displayed on the display unit.

The advantage of the present invention provides the three-way connector, the one end of the three-way connector is connected to the placing tube, and another end of the three-way connector is allowed the endoscope to enter. The endoscope is then inserted into the placing tube. Meanwhile, the images are captured and shown on the display unit. If needed, the air/fluid can be pumped into the natural tract of the human body through the three-way connector to inflate the cavity of the human body for have a better vision. As a result, the placing tube can be placed to the target position, and its position can be confirmed more easily.

The accompanying drawings are incorporated in and constitute a part of this application and, together with the description, serve to explain the principles of the disclosure in general terms. Like numerals refer to like parts throughout the disclosure.

### BRIEF DESCRIPTION OF THE DRAWINGS

The objects, spirits, and advantages of the preferred embodiments of the present disclosure will be readily understood by the accompanying drawings and detailed descriptions, wherein:

FIG. 1 illustrates a schematic exploded view of a system of a first embodiment of the present invention;

FIG. 2 illustrates a schematic assembly view of the system of the first embodiment of the present invention;

FIG. 3 illustrates a schematic operation view of the system of the first embodiment of the present invention;

FIG. 4 illustrates a schematic block diagram of an endoscope of the system of the first embodiment of the present invention;

FIG. 5A illustrates a schematic exploded view of the bending section of the first embodiment of the present invention;

FIG. 5B illustrates a schematic exploded view of another bending section of the first embodiment of the present invention;

FIG. 5C illustrates a schematic assembly view of the bending section of the first embodiment of the present invention;

FIG. 6A illustrates a schematic exploded view of a bending section of the second embodiment of the present invention;

FIG. 6B illustrates a schematic exploded bottom view of the bending section of the second embodiment of the present invention;

FIG. 6C illustrates a schematic assembly view of the bending section of the second embodiment of the present invention;

FIG. 6D illustrates a schematic top view of another bending section of the second embodiment of the present invention;

FIG. 6E illustrates a schematic assembly view of the bending section of the second embodiment of the present invention;

FIG. 7A illustrates a schematic exploded view of a bending section of the third embodiment of the present invention;

FIG. 7B illustrates a schematic exploded bottom view of the bending section of the third embodiment of the present invention;

FIG. 7C illustrates a schematic assembly view of the bending section of the third embodiment of the present invention; and

FIG. 8 illustrates a flow chart of an embodiment of the present invention.

### DETAILED DESCRIPTION

In order to describe in detail the technical content, structural features, achieved objectives and effects of the instant application, the following detailed descriptions are given in conjunction with the drawings and specific embodiments. It should be understood that these embodiments are only used to illustrate the application and not to limit the scope of the instant application.

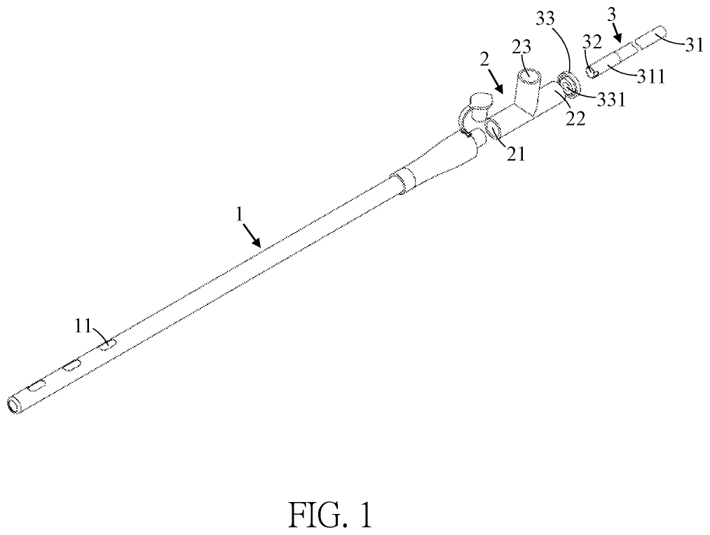

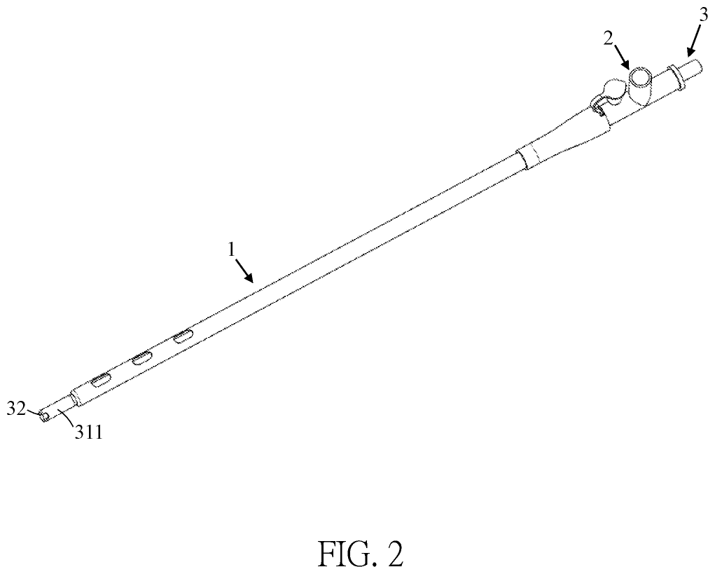

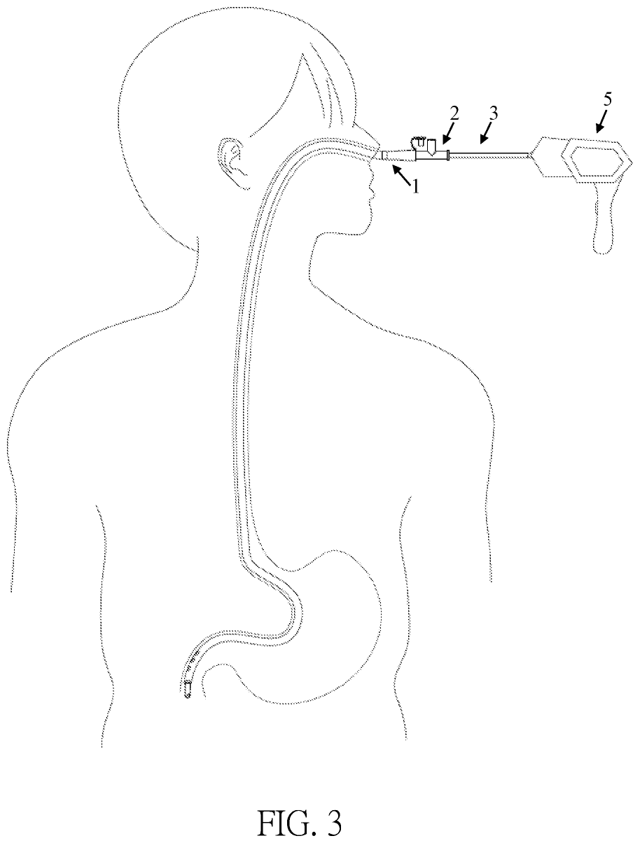

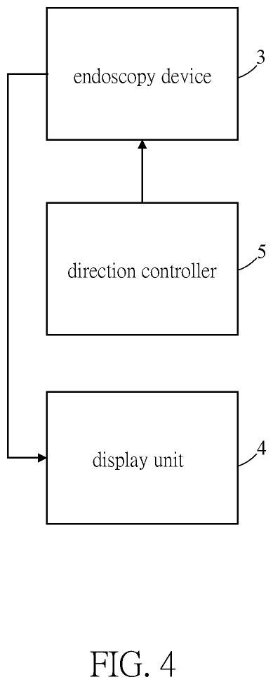

With reference to FIG. 1, FIG. 2, FIG. 3, and FIG. 4, which illustrate a schematic exploded view of a system of a first embodiment of the present invention, a schematic assembly view of the system of the first embodiment of the present invention, a schematic operation view of the system of the first embodiment of the present invention, and a schematic block diagram of an endoscope of the system of the first embodiment of the present invention. As shown in the figures, the system for aiding placement of a nasogastric tube or a nasoduodenal tube of the present invention includes a placing tube 1, a three-way connector 2, an endoscope 3, and a display unit 4, which are described in detail as following.

For the first embodiment, the placing tube 1 is a nasogastric tube (NG Tube) or a nasoenteric tube (NE Tube) that has many side holes 11. It is for people who have difficulties in eating, but not limited thereto.

The three-way connector 2 includes a first port 21, a second port 22 and a third port 23. The first port 21 and the second port 22 are on the two opposite end of the channel, while the third port 23 is adjacent to the first port 21. The first port 21 is connected to the proximal end of the placing tube 1, the second port 22 is a port for the endoscope 3 to pass through, and the third port 23 is for air or fluid injection, but not limited thereto.

The endoscope 3 includes an insertion tube 31 and an image-capturing device 32. For the first embodiment, one end of the insertion tube 31 is a bending section 311, and the image-capturing device 32 is located at the distal end of the bending section 311, but not limited thereto. Further, the endoscope 3 has a light source (not shown in figure), which emits a light into a natural tract of the human body, so as to let the image-capturing device 32 capture images and display the captured images on the display unit 4. The images captured by the image-capturing device contain information of internal organs/tissues reflected by the light.

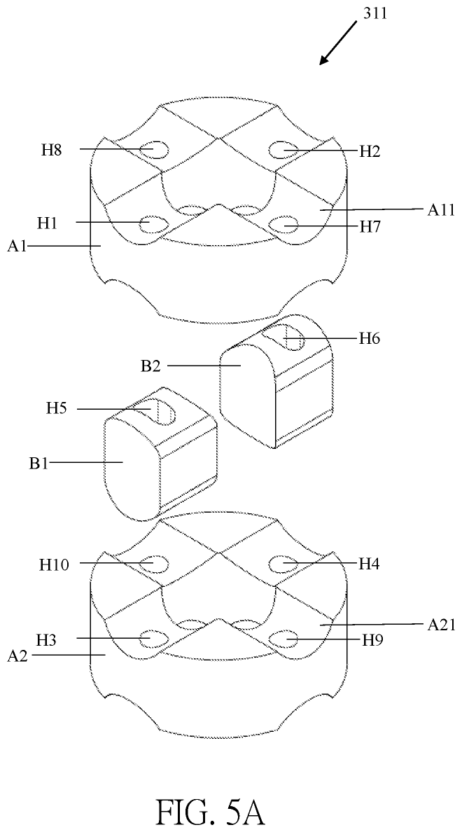

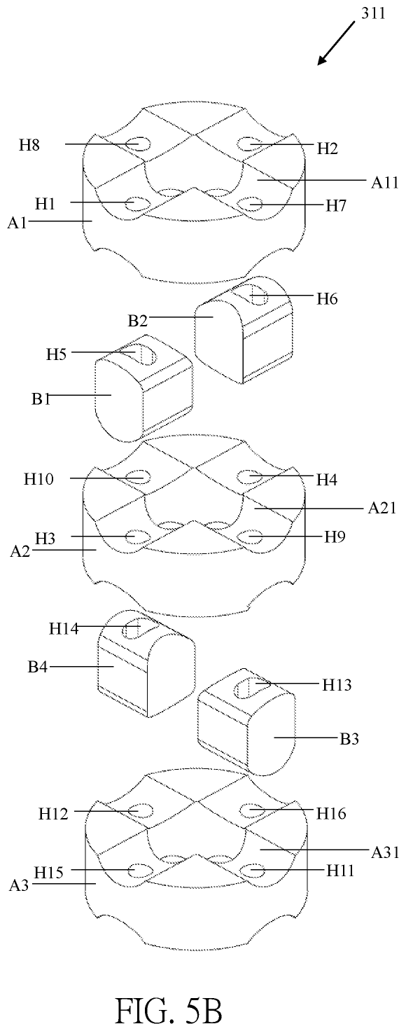

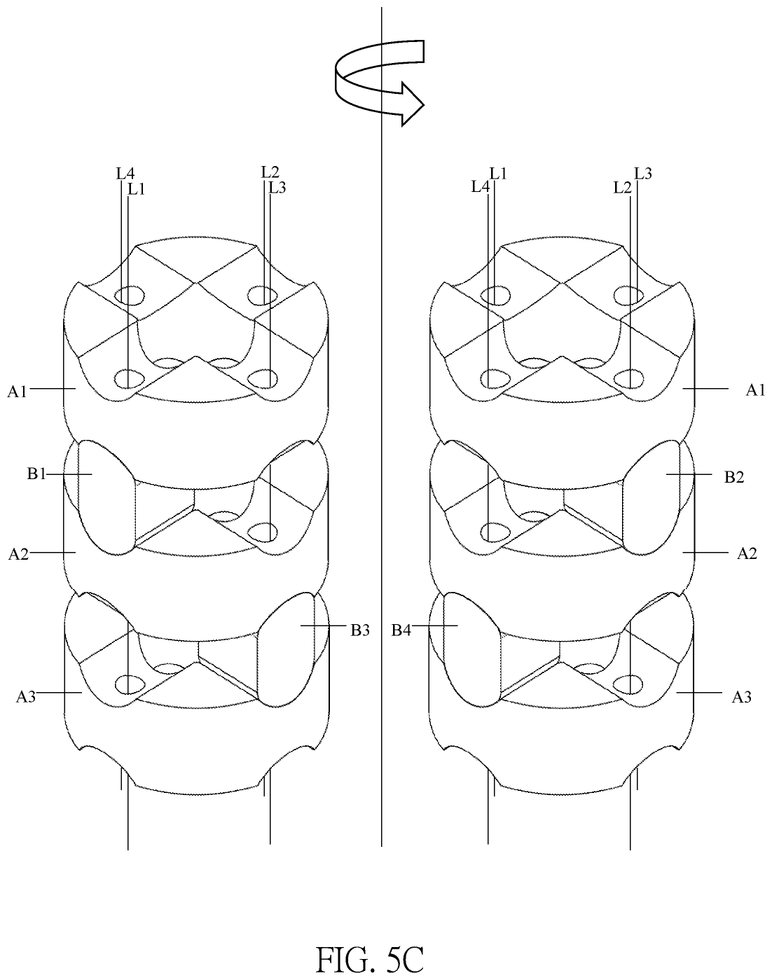

Referring to FIG. 5A, which illustrates a schematic exploded view of the bending section of the first embodiment of the present invention. According to FIG. 5A, the bending section 311 includes a first body A1, a second body A2, a first pivot element B1, and a second pivot element B2. The following describes the bending section in detail.

The first body A1 has eight concave shapes A11, which are located in the four directions on the top and the bottom surfaces of the first body A1, and there are a first hole H1, a second hole H2, a seventh hole H7 and an eighth hole H8 located in middle of concave shapes A11.

According to the present embodiment, the second body A2 is the same as the first body A1, but not limited thereto. The second body A2 has eight concave shapes A21, which are located in the four directions on the top and the bottom surfaces of the second body A2, and there are a third hole H3, a fourth hole H4, a ninth hole H9, and a tenth hole H10 located in middle of concave shapes A21.

The first pivot element B1 has a fifth hole H5 corresponding to the first hole H1 in structure. One surface of the first pivot element B1 fits to the concave shape A11, and another surface of the first pivot element B1 fits to the concave shape A21. The fifth hole H5 is corresponding to the third hole H3 as well so that the first pivot element B1 is between the first body A1 and the second body A2, connecting the first body A1 and the second body A2.

The second pivot element B2 is the same as the first pivot element B1 in structure. The second pivot element B2 has a sixth hole H6. One surface of the second pivot element B2 fits to concave shape A11 so that the sixth hole H6 corresponds to the second hole H2; another surface of the second pivot element B2 fits to concave shape A21 so that the sixth hole H6 corresponds to the fourth hole H4. As it can be seen that the second pivot element B2 is between the first body A1 and the second body A2, connecting the first body A1 and the second body A2.

The first wire L1 is put through the first hole H1, the third hole H3 and the fifth hole H5, and the second wire L2 is put through the second hole H2, the fourth hole H4, and the sixth hole H6 so that the first body A1 and the second body A2 are connected to each other via the first pivot element B1 and the second pivot element B2.

Accordingly, the third wire L3 is put through the seventh hole H7 and the third hole H9, and the fourth wire L4 is put through the eighth hole H8 and the tenth hole H10. As a result, the axis of the bending section 311 will move towards the third wire L3 when pulling the third wire L3, while the axis of the bending section 311 moves towards the fourth wire L4 when pulling the fourth wire L4.

In relation to FIG. 5B and FIG. 5C, which illustrate a schematic exploded view of another bending section of the first embodiment of the present invention and a schematic assembly view of the bending section of the first embodiment of the present invention. As it can be seen, by assembling more bodies together, the length of the bending section can be extended, and the bending angle can be increased. The original two-way steering structure can be transformed into a four-way steering structure, and the new structure enables the axis of the bending section to bend forwards the wires pulled.

For the embodiment, the bending section 311 further has a third body A3, a third pivot element B3 and a fourth pivot element B4. The third body A3 includes eight concave shapes A31, which are located in the four directions on the top and the bottom surfaces of the third body A3. There are an eleventh hole H11, a twelfth hole H12, a fifteenth hole H15 and a sixteenth hole H16 located in the middle of concave shapes A31, which means the third body A3 is the same as the first body A1 and the second body A2, but not limited thereto.

The third pivot element B3 has a thirteenth hole H13. One surface of the third pivot element B3 correspondingly fits to concave shape A21 so that the thirteenth hole H13 and the ninth hole H9 are connected to each other. Another surface of the third pivot element B3 fits to the concave shape A31 so that the thirteenth hole H13 and the eleventh hole H11 are connected to each other. Therefore, the third pivot element B3 is between the second body A2 and the third body A3, meaning that the third body A3 and the second body A2 are connected to each other via the third pivot element B3.

The fourth pivot element B4 has a fourteenth hole H14 corresponding to the tenth hole H10, one surface of the fourth pivot element B4 fits to the concave shape A21. Another surface of the fourth pivot element B4 fits to the concave shape A31, and the fourteenth hole H14 is corresponding to the twelfth hole H12 as well. Therefore, the fourth pivot element B4 is between the second body A2 and the third body A3, meaning that the third body A3 and the second body A2 are connected to each other via the fourth pivot element B4.

Equally, the first wire L1 is through the first hole H1, the third hole H3, the fifth hole H5, and the fifteenth hole H15; the second wire L2 penetrates through the second hole H2, the fourth hole H4, the sixth hole H6, and the sixteenth hole H16. So that the first body A1, the second body A2 and the third body A3 are connected to each other. Therefore, the axial directions of the bending section 311 will move toward the first wire L1 when pulling the first wire L1; on the other hand, the axial directions of the bending section 311 will move toward the second wire L2 when pulling the second wire L2.

Accordingly, the third wire L3 is through a seventh hole H7, a ninth hole H9, an eleventh hole H11, and a thirteenth hole H13; on the other hand, the fourth wire L4 penetrates through an eighth hole H8, a tenth hole H10, a twelfth hole H12, and a fourteenth hole H14. Therefore, the axial directions of the bending section 311 will move towards the third wire L3 when pulling the third wire L3, and the axial directions of the bending section 311 will move towards the fourth wire L4 when pulling the fourth wire L4.

Aforesaid embodiments are part of the embodiments of the present invention. The numbers of the body and the pivot element may not be limited and depend on how many the bodies and the pivot elements are connected. Further, the concave shape on the body is connected to one surface of the pivot element. That is, the shape of the concave part of the concave shape basically matches the shape of the convex part of the pivot element. Additionally, one body and two pivot elements should be added to the bending section together. The first layer of the pivot elements and the second layer of the pivot elements can be placed staggeredly to control the bending section more smoothly, but not limited thereto.

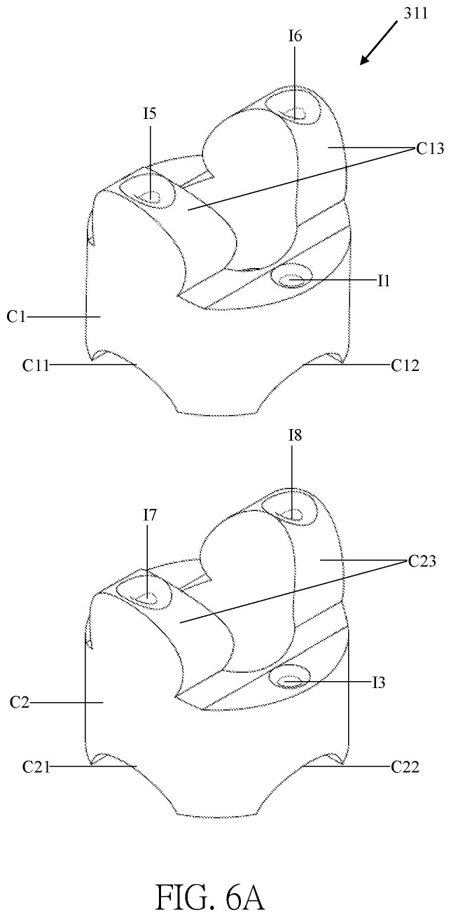

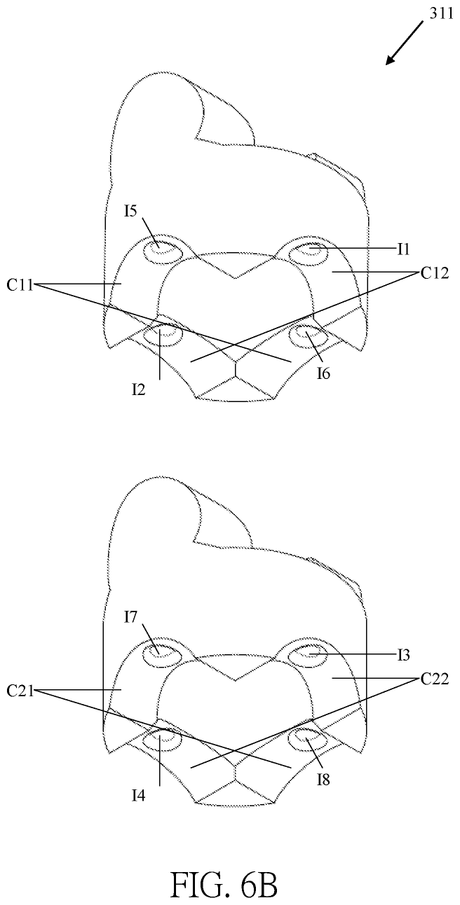

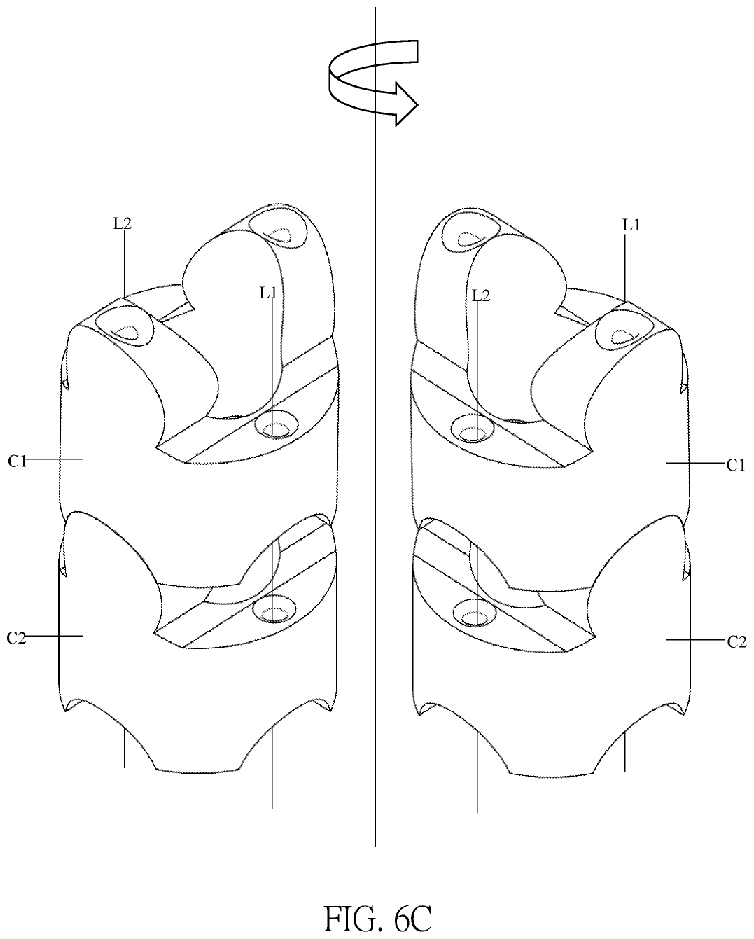

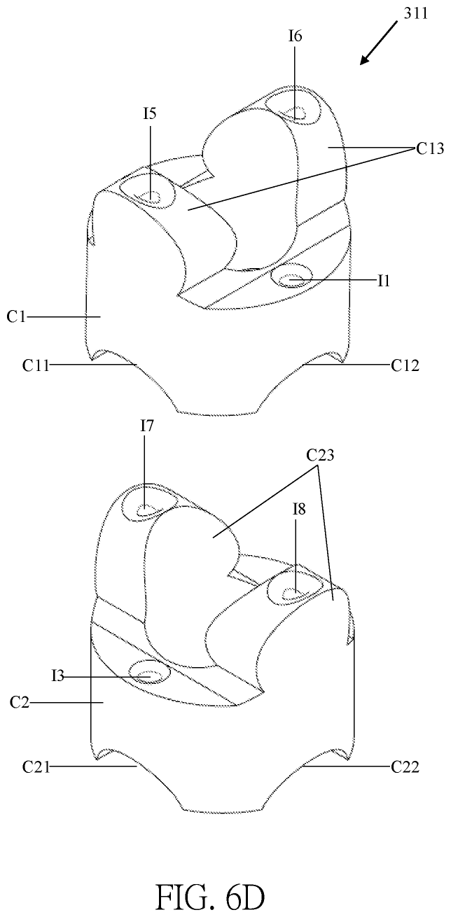

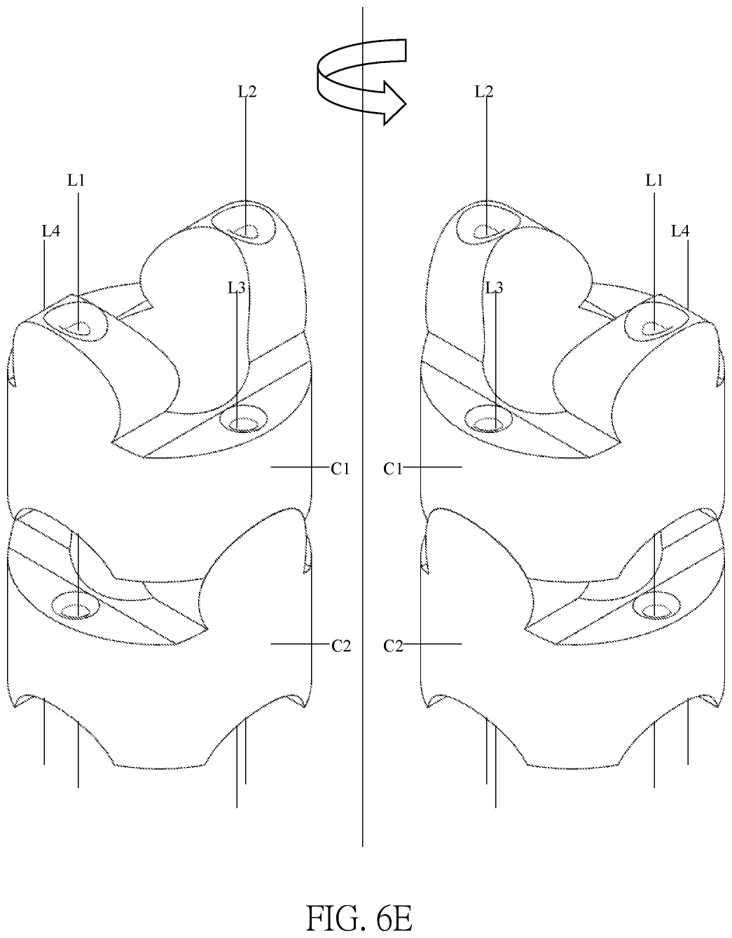

In regard to FIG. 6A, FIG. 6B and FIG. 6C, which illustrate a schematic exploded view of a bending section of the second embodiment of the present invention, a schematic exploded bottom view of the bending section of the second embodiment of the present invention and a schematic assembly view of the bending section of the second embodiment of the present invention. According to those figures, the bending section 311 of the second embodiment includes a first body C1 and a second body C2, and it will be described in detail as follows.

The first body C1 has two concave shapes C11, two concave shapes C12 and two convex shapes C13. Concave shapes C11 and concave shapes C12 are located on a first surface of the first body C1. The concave shape C12 has a first hole I1 and a second hole I2. The first convex shapes C13 are located on a second surface of the first body C1. The position of convex shape C13 on the second surface corresponds to the position of concave shape C11 on the first surface. For the second embodiment, convex shape C13 is orthographically projected on the first surface, and it just overlaps with the orthographic projection of concave shape C11 on the first surface, but not limited thereto.

Based on the second embodiment, the second body C2 is the same as the first body C1, but not limited hereto. The second body C2 has two concave shapes C21, two concave shapes C22 and two convex shapes C23. The concave shapes C21 and the concave shapes C22 are located on a first surface of the second body C2. The concave shape C22 has a third hole I3 and a fourth hole I4. The convex shape C23 is located on a second surface of the second body C2, a position wherein the second convex shape C23 on the second surface is corresponding to the position of concave shape C21 on the first surface. The second convex shape C23 fits into the concave shape C11 in order to let the third hole I3 correspond to the first hole I1 and the fourth hole I4 correspond to the second hole I2. For the second embodiment, the convex shape C23 is orthographically projected on the first surface, it just overlaps with the orthographic projection of the concave shape C21 on the first surface, but not limited thereto. The convex shape C23 fits into the concave shape C11 in order to let the third hole I3 correspond to the first hole I1 and the fourth hole I4 correspond to the second hole I2.

More, a first wire L1 is put through the first hole I1 and the third hole I3; and a second wire L2 is put through the second hole I2 and the fourth hole I4. As a result, the axial directions of the bending part 311 may deflect towards the first wire L1 when the first wire L1 is pulled; on the other hand, the axial directions of the bending part 311 may deflect toward the second wire L2 when the second wire L2 is pulled.

If the arrangements of FIG. 6C adopt the overlapping arrangements of FIG. 5B and FIG. 5C, the bending section shall be longer, which results in smoothness and a larger bending angle. Additionally, the staggered setting converts the original two-way steering structure into a four-way steering structure.

When the original two-way steering structure converting into a four-way steering structure, a schematic top view of another bending section of the second embodiment of the present invention and a schematic assembly view of the bending section of the second embodiment of the present invention are illustrated in FIG. 6D and FIG. 6E. As shown in the figures, the concave shape C11 has a fifth hole I5 and a sixth hole I6, which pass from the two convex shapes C13 to the two concave shapes C11. The concave shape C21 has a seventh hole I7 and an eighth hole I8, which pass from the two convex shapes C23 to the two concave shapes C21.

In the second embodiment, the first body C1 and the second body C2 adopt a staggered setting, that is, the convex shape C23 is beneath the concave shape C12 so as to make the seventh hole I7 correspond to the second hole I2, the eighth hole I8 correspond to the first hole I1, and the third hole I3 and the fourth hole I4 correspond to the concave shape C11. Furthermore, the first wire L1 goes through the fifth hole I5 and the third hole I3, the second wire L2 goes through the sixth hole I6 and the fourth hole I4, the third wire L3 goes through the first hole I1 and the eighth hole I8, and the fourth wire L4 passes through the second hole I2 and the seventh hole I7. In this way, the axial direction of the bending part 311 will move towards the first wire L1 when the first wire L1 is pulled; on the other hand, the axial direction of the bending part 311 will move towards the second wire L2 when the second wire L2 is pulled.

As it can be seen in the second embodiment, the bodies and parts for bending and wire control can all be properly staggered or stacked up. The original two-way steering structure can therefore be transformed into a four-way steering structure, and the axis direction of the entire structure will move towards a specific wire when that wire is pulled.

Aforesaid embodiment is part of applications, the concave shape and the convex shape match each other, that is, the structure of the concave shape matches the structure of the convex shape, but not limited thereto.

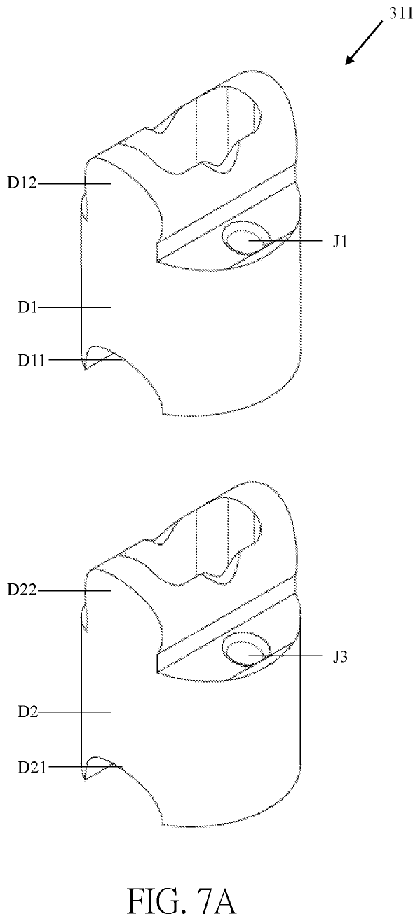

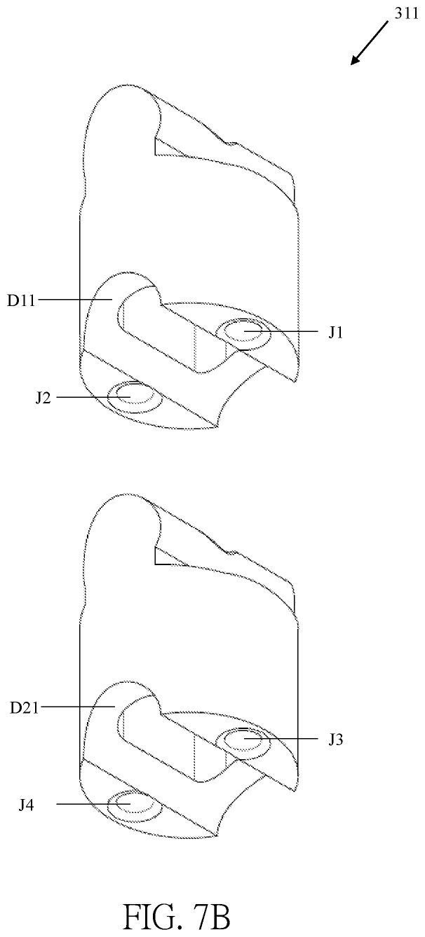

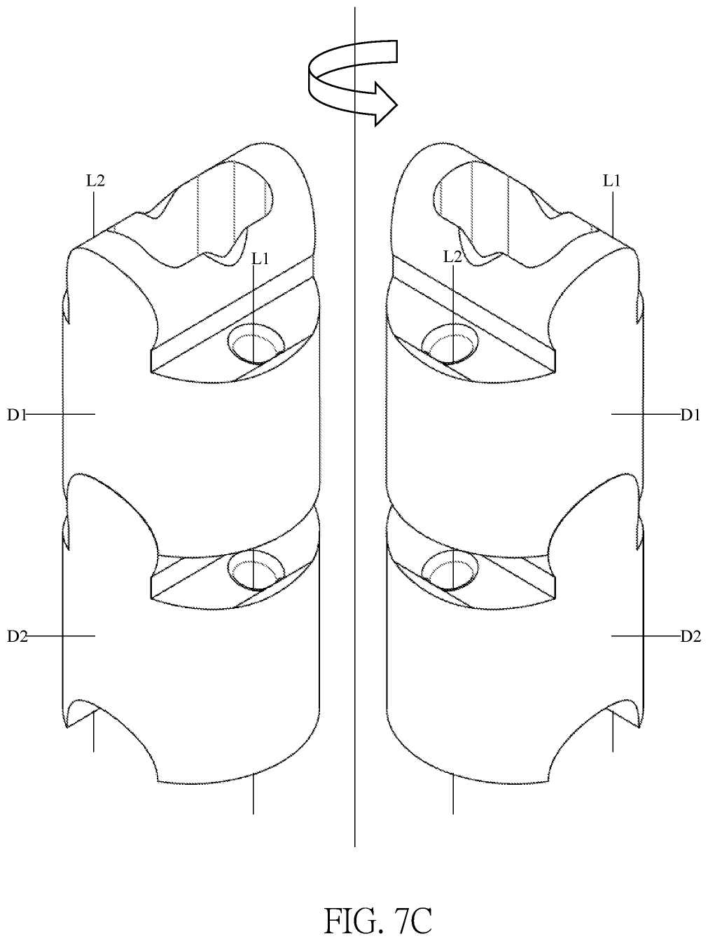

Please refer to FIG. 7A, FIG. 7B and FIG. 7C, which illustrate a schematic exploded view of a bending section of the third embodiment of the present invention, a schematic exploded bottom view of the bending section of the third embodiment of the present invention, and a schematic assembly view of the bending section of the third embodiment of the present invention. As shown in the figures, the bending section 311 of the third embodiment includes a first body D1 and a second body D2, and it is described in detail as follows.

A first body D1 has a concave shape D11, a convex shape D12, a first hole J1, and a second hole J2. The concave shape D11 is located on the first surface of the first body D1. The convex shape D12 is located on the second surface of the first body D1. The convex shape D12 on the second surface is corresponding to the concave shape D11 on the first surface. For the third embodiment, the convex shape D12 is orthographically projected on the first surface, and it just overlaps with the orthographic projection of the concave shape D11 on the first surface, but not limited thereto. The first hole J1 is at one side of the first body D1; on the other hand, the second hole J2 is at another side of the first body D1. Therefore, for the third embodiment, the first hole J1 and the second hole J2 are not overlapped with the concave shape D11 and the convex shape D12 with no any limitation.

Based on the second embodiment, the second body D2 is the same as the first body D1, but without any limitation. The second body D2 includes a concave shape D21, a convex shape D22, a third hole J3, and a fourth hole J4. The concave shape D21 is located on the first surface of the second body D2. The second convex shape D22 is located on the second surface of the second body D2. The convex shape D22 on the second surface is corresponding to the concave shape D21 on the first surface. For the third embodiment, the convex shape D22 is orthographically projected on the first surface, and it just overlaps with the orthographic projection of the second concave shape D12 on the first surface, but not limited thereto. The third hole J3 is at one side of the second body D22, and the fourth hole J4 is at another side of the second body D22. Therefore, for the third embodiment, the third hole J3 and the fourth hole J4 are not overlapped with the concave shape D12 and the convex shape D22 with no any limitation. The convex shape D22 fits to the concave shape D21 in order to let the third hole J3 be aligned with the first hole J1, and let the fourth hole J4 be aligned with the second hole J2.

A first wire L1 passes through the first hole J1 and the third hole J3, and a second wire L2 passes through the second hole J2 and the fourth hole J4. In this way, the axial direction of the bending section 311 will move towards the first wire L1 when the first wire L1 is pulled; on the other hand, the axial direction of the bending section 311 will move towards the second wire L2 when the second wire L2 is pulled.

Aforesaid embodiment is only part of the applications. The number of the body is not limited and can be assembled depending on the needs. The concave shape and the convex shape match each other, that is, the structure of the concave shape matches the structure of the convex shape, but not limited thereto.

The assembly of aforementioned bodies and pivot elements and the assembly of the bodies require only wires to pull and control the axial direction of the bending section 311. Such methods can be easily handled, and the bodies and/or the pivot elements can be concatenated depending on the needs. The entire structure is very flexible, and the deflection angle can become wider by adjusting the number of the bodies and/or the pivot elements. So, it has many advantages compared with the prior arts.

The present invention further has a cover 33 with a hole structure 331. After combining the hole structure 331 with the cover 33, the endoscope 3 covers the port second port 22 in order to protect the air or fluid from draining out from the second port 22. Another option is to integrate the cover 33 and the endoscope 3 into one device, but not limited thereto.

The display unit 4 is connected with the endoscope 3 for receiving and displaying images from the endoscope 3. Those images help an operator view where the placing tube 1 is in the human body, but without any limitation.

The present invention further has a direction controller 5 in the endoscope 3, so that the wires of the bending section 311 of the endoscope 3 can be controlled for moving the axial directions of the distal end of the endoscope 3. As a result, the placing tube (1) can be controlled to reach to the target position, but not limit thereto.

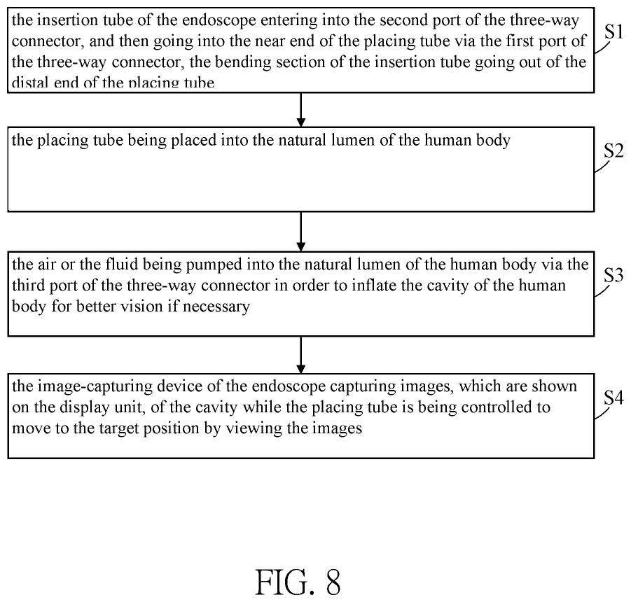

Please refer to FIG. 8, which illustrates a flow chart of an embodiment of the present invention. According to FIG. 8, the method for aiding placement of nasogastric/nasoduodenal tubes of the present invention includes the steps as follows.

Step (S1) is of: the insertion tube 31 of the endoscope 3 entering the second port 22 of the three-way connector 2, and then going into the proximal end of the placing tube 1 via the first port 21 of the three-way connector 2, the bending section 311 of the insertion tube 31 sticking out of the distal end of the placing tube 1;

Step (S2) is of: the placing tube 1 being placed into a natural tract of the human body;

Step (S3) is of: air or fluid being pumped into a natural tract of the human body via the third port (23) of the three-way connector (2) in order to inflate the cavity of the human body for better vision if necessary;

Step (S4) is of: the image-capturing device 32 of the endoscope 3 capturing images, which are shown on the display unit 4 while the placing tube (1) can be controlled and placed to the target position by viewing the images.

As shown in step (S1), the insertion tube 31 of the endoscope 3 goes into the first port 21 of the three-way connector 2, and then the bending section 311 of the insertion tube 31 goes out of the distal end of the placing tube 1. For the present invention, the placing tube 1 is, for example, a nasogastric tube. Further, after combining the hole structure 331 with the cover 33 and after the endoscope 3 enters the second port 22 of the three-way connector 2, the endoscope 3 will be able to cover the second port 22.

As shown in step (S2), the placing tube 1 with the endoscope 3 goes into a natural tract of the human body, wherein the natural tract of the human body, in this embodiment, is a natural cavity inside the human body, and the natural cavity can be a nasal cavity or an oral cavity. A light source emitting light to the natural tract of the human body can be applied to capture clearer images. The image-capturing device 32 captures the images that contain information of the internal organs/tissues reflected by the light. The images will then be shown on the display unit 4, but not limited thereto.

As shown in step (S3), there may be narrow cavities in human body, which makes it difficult to capture clear images for the endoscope 3. If this occurs, pumping the air/fluid into the cavity via the third port 23 of the three-way connector 2 to inflate the cavity so that the better images can be obtained by the endoscope 3.

As shown in step (S4), the image-capturing device 32 of the endoscope 3 captures the images, which are shown on the display unit 4 immediately while the placing tube (1) is controlled to be placed to the target position by viewing the images. As an example for the embodiment, the target position is a digestive tract, but with no limitations. Further, the direction controller 5 controls the axial direction of the distal end of the endoscope 3 to guide the placing tube 1 to the target position. After the placing tube 1 reaches the target position, the three-way connector 2 and the endoscope 3 can be removed, and then the placing tube 1 can be used as a feeding tube or other purposes.

As a conclusion, the present invention discloses a system for aiding placement of a nasogastric tube or a nasoduodenal tube and method for using the same, more particularly a system for helping to place nasogastric/nasoenteric tubes and a method thereof. That is, the three-way connector is connected to the placing tube for the endoscope going into the placing tube. Then the images of the natural tract of the human body are obtained by the endoscope in order to control the placing tube to go to the target position. It highly decreases the failure rate during intubation and improve safety.

Although the disclosure has been disclosed and illustrated with reference to particular embodiments, the principles involved are susceptible for use in numerous other embodiments that will be apparent to a person having ordinary skill in the art. This disclosure is, therefore, to be limited only as indicated by the scope of the appended claims.

What is claimed is:

1. A system for aiding placement of a nasogastric tube or a nasoduodenal tube, comprising:
a placing tube (1);
a three-way connector (2) that has a first port (21), a second port (22) and a third port (23) with the first port (21) on the opposite end to the second port (22) and the third port (23) adjacent to the first port (21); wherein said first port (21) is connected with the proximal end of the placing tube (1) and the third port (23) designed for air/fluid injection;
an endoscope (3) that has an insertion tube (31) and an image-capturing device (32) with the distal end of the insertion tube being a bending section (311) and the image-capturing device (32) being set at the front end of the bending section (311); and
a display unit (4) that is connected to the endoscope (3);
wherein the endoscope (3) is inserted into the second port (22) and then through the first port (21) of the three-way connector (2) to the distal end of the insertion tube (1), with the bending section (311) going out of the distal end of the placing tube (1); after the placing tube (1) is inserted into a natural tract of the human body, pump air or fluid into the natural tract via the third port (23) of the three-way connector (2) in order to inflate a cavity of the human body, if needed, thus the image-capturing device (32) capturing images in the natural tract, which are then instantaneously shown on the display unit (4), the placing tube (1) being placed into the target position by viewing the lumen images, after that, the endoscope (3) and the three-way connector (2) being removed from the placing tube (1).

2. The system for aiding placement of a nasogastric tube or a nasoduodenal tube, according to claim 1, further comprising a cover (33) that has a hole structure (331), the endoscope (3) being combined with the cover (33) through the hole structure (331) so that the cover (33) covers the second port (22) of the three-way connector (2) when the endoscope (3) goes through the three-way connector (2).

3. The system for aiding placement of a nasogastric tube or a nasoduodenal tube, according to claim 1, further comprising a direction controller (5), which controls the axial direction of the distal end of the endoscope (3), so that the endoscope (3) is controlled to let the placing tube (1) be guided into the target position.

4. The system for aiding placement of a nasogastric tube or a nasoduodenal tube, according to claim 1, wherein the endoscope (3) has a light source that emits a light to a natural tract of the human body.

5. The system for aiding placement of a nasogastric tube or a nasoduodenal tube, according to claim 4, wherein the images captured by the image-capturing device (32) contain information of internal organs/tissues reflected by the light and are then displayed on the display unit (4).

6. The system for aiding placement of a nasogastric tube or a nasoduodenal tube, according to claim 1, wherein the bending section (311) comprises:
a first body (A1), having a plurality of concave shapes (A11) located on the top/bottom surfaces of the first body (A1) with some of concave shapes (A11) having a first hole (H1) and a second hole (H2) respectively;
a second body (A2), having a plurality of concave shapes (A21) located on the top/bottom surfaces of the second body (A2) with some of the concave shapes (A21) having a third hole (H3) and a fourth hole (H4) respectively;
a first pivot element (B1), having a fifth hole (H5) corresponding to the first hole (H1), one surface of the first pivot element (B1) being placed on the concave shape (A11) of the first body (A1), another surface of the first pivot element (B1) being placed on the concave shape (A21) of the second body (A2), and the fifth hole (H5) being aligned with the third hole (H3) so that the first body (A1) and the second body (A2) are connected to each other via the first pivot element (B1);
a second pivot element (B2), having a sixth hole (H6) corresponding to the second hole (H2), one surface of the second pivot element (B2) being placed on the concave shape (A11) of the first body (A1), another surface of the first pivot element (B1) being placed on the concave shape (A21) of the second body (A2), and the sixth hole (H6) being aligned with the fourth hole (H4) so that the first body (A1) and the second body (A2) are connected to each other via the second pivot element (B2);
a first wire (L1), passed through the first hole (H1), the third hole (H3), and the fifth hole (H5); and
a second wire (L2), passed through the second hole (H2), the fourth hole (H4), and the sixth hole (H6);
wherein the bending section (311) is assembled.

7. The system for aiding placement of a nasogastric tube or a nasoduodenal tube according to claim 6, wherein the concave shapes (A11) of the first body (A1) further have a seventh hole (H7) and an eighth hole (H8), and the second concave shapes (A21) of the second body (A2) further have a ninth hole (H9) and a tenth hole (H10), the seventh hole (H7) being corresponding to the ninth hole (H9), the eighth hole (H8) being corresponding to the tenth hole (H10).

8. The system for aiding placement of a nasogastric tube or a nasoduodenal tube according to claim 7, wherein the bending section (311) has a third wire (L3) and a fourth wire (L4), with the third wire (L3) passed through the seventh hole (H7) and the ninth hole (H9), and the fourth wire (L4) passed through the eighth hole (H8) and the tenth hole (H10), the axial direction of the bending section (311) moving towards the third wire (L3) when the third wire (L3) is pulled, or the axial direction of the bending section (311) moving towards a direction of the fourth wire (L4) when the fourth wire (L4) is pulled.

9. The system for aiding placement of a nasogastric tube or a nasoduodenal tube according to claim 7, wherein the bending section (311) further comprises a third body (A3), a third pivot element (B3), and a fourth pivot element (B4), the third body (A3) having at least eight concave shapes (A31), which are located in the top/bottom surfaces of the third body (A3) and have an eleventh hole (H11) and a twelfth hole (H12), the third pivot element (B3) having a thirteenth hole (H13) corresponding to the ninth hole (H9) with one surface of the third pivot element (B3) placed into the concave shape (A21) of the second body (A2), and another surface of the third pivot element (B3) placed into the concave shape (A31) of the third body (A3), and the thirteenth hole (H13) being corresponding to the eleventh hole (H11) so that the third body (A3) and the second body (A2) are connected to each other via the third pivot element (B3); the fourth pivot element (B4) having a fourteen hole (H14) corresponding to the tenth hole (H10), one surface of the fourth pivot element (B4) being placed on the concave shape (A21) of the second body (A2), another surface of the fourth pivot element (B4) being matched up the concave shape (A31) of the third body (A3), and the fourteenth hole (H14) corresponding to the twelfth hole (H12), thus the fourth pivot element B4 being between the second body A2 and the third body A3, which means the third body (A3) and the second body (A2) being connected to each other via the fourth pivot element (B4).

10. The system for aiding placement of a nasogastric tube or a nasoduodenal tube, according to claim 9, wherein the concave shape (A31) of the third body (A3) has a fifteenth hole (H15) and a sixteenth hole (H16), the first wire (L1) passed through the first hole (H1) of the concave shape (A11), the third hole (H3) of the concave shape (A21), the fifth hole (H5) of the first pivot element (B1), and the fifteenth hole (H15) of the concave shape (A31), the second wire (L2) passed through the second hole (H2) of the concave shape (A11), the fourth hole (H4) of the concave shape (A21), the sixth hole (H6) of the second pivot element (B2), and the sixteenth hole (H16) of the concave shape (A31), the axial direction of the bending section (311) moving towards the first wire (L1) when the first wire (L1) is pulled, or the axial direction of the bending section (311) moving towards the second wire (L2) when the second wire (L2) is pulled.

11. The system for aiding placement of a nasogastric tube or a nasoduodenal tube, according to claim 6, wherein the bending section (311) further comprises a third wire (L3) and a fourth wire (L4), the third wire (L3) passed through a seventh hole (H7) of the concave shape (A11), a ninth hole (H9) of the concave shape (A21), an eleventh hole (H11) of a concave shape (A31), and a thirteenth hole (H13) of a third pivot element (B3), and the fourth wire (L4) passed through an eighth hole (H8) of the concave shape (A11), a tenth hole (H10) of the concave shape (A21), a twelfth hole (H12) of the concave shape (A31), and a fourteenth hole (H14) of the fourth pivot element (B4), the axial direction of the bending section (311) moving towards the third wire (L3) when the third wire (L3) is pulled, or the axial direction of the bending section (311) moving towards the fourth wire (L4) when the fourth wire (L4) is pulled.

12. The system for aiding placement of a nasogastric tube or a nasoduodenal tube, according to claim 1, wherein the bending portion (311) comprises:
a first body (C1), having at least two concave shapes (C11), at least two concave shapes (C12) and at least two convex shapes (C13), the concave shapes (C11) and the concave shapes (C12) being located on a first surface of the first body (C1), the concave shapes (C12) having a first hole (I1) and a second hole (I2), the convex shape (C13) being located on a second surface of the first body (C1), a position where the convex shape (C13) is on the second surface being corresponding to a position where the concave shapes (C11) is on the first surface;
a second body (C2), having at least two concave shapes (C21), at least two concave shapes (C22) and at least two convex shapes (C23), the concave shapes (C21) and the concave shapes (C22) being located on a first surface of the second body (C2), while the concave shapes (C22) having a third hole (I3) and a fourth hole (I4), the convex shapes (C23) being located on a second surface of the second body (C2), the position of the convex shape (C23) on the second surface being corresponding to the position of the concave shape (C21) on the first surface, the convex shape (C23) fitting into the concave shape (C11) in order to let the third hole (I3) be corresponding to the first hole (I1), and the fourth hole (I4) be corresponding to the second hole (I2);
a first wire (L1), passed through the first hole (I1) and the third hole (I3); and
a second wire (L2), passed through the second hole (I2) and the fourth hole (I4);
wherein the axial direction of the bending section (311) being moved towards the first wire (L1) when the first wire (L1) is pulled, or the axial direction of the bending section (311) being moved towards the second wire (L2) when the second wire (L2) is pulled.

13. The system for aiding placement of a nasogastric tube or a nasoduodenal tube, according to claim 12, wherein the concave shape (C11) has a fifth hole (I5) and a six hole (I6), which pass from the convex shapes (C13) to the concave shapes (C11), the concave shapes (C21) having a seventh hole (I7) and an eighth hole (I8), which penetrate through the convex shapes (C23) to the concave shapes (C21).

14. The system for aiding placement of a nasogastric tube or a nasoduodenal tube, according to claim 13, wherein the bending section (311) comprises a third wire (L3) and a fourth wire (L4), the third wire (L3) passed through the fifth hole (I5) and the seventh hole (I7), the fourth wire (14) passed through the sixth hole (I6) and the eighth hole (I8).

15. The system for aiding placement of a nasogastric tube or a nasoduodenal tube, according to claim 1, wherein the bending section (311) comprises:
a first body (D1), having a concave shape (D11), a first convex shape (D12), a first hole (J1), and a second hole (J2), the concave shape (D11) being placed on a first surface of the first body (D1), the convex shape (D12) being placed at a second surface of the first body (D1), the convex shape (D12) on the second surface being corresponding to the concave shape (D11) on the first surface, the first hole (J1) being at a side of the first body (D1), the second hole (J2) being at another side of the first body (D1);
a second body (D2), having a concave shape (D21), a convex shape (D22), a third hole (J3), and a fourth hole (J4), the concave shape (D21) being placed on a first surface of the second body (D2), the convex shape (D22) being placed on a second surface of the second body (D2), the convex shape (D22) on the second surface of the second body (D2) being corresponding to the concave shape (D21) on the first surface of the second body (D2), the third hole (J3) being at a side of the second body (D22), the fourth hole (J4) being at another side of the second body (D22), the convex shape (D22) being matched for the concave shape (D11) of the first body (D1) in order to let the third hole (J3) correspond to the first hole (J1), and the fourth hole (J4) correspond to the second hole (J2);
a first wire (L1), passed through the first hole (J1) and the third hole (J3); and
a second wire (L2), passed through the second hole (J2) and the fourth hole (J4);
wherein an axial direction of the bending section (311) is deflected towards the first wire (L1) when the first wire (L1) is pulled, or the axial direction of the bending section (311) is deflected towards the second wire (L2) when the second wire (L2) is pulled.

16. A method for aiding placement of a nasogastric tube or a nasoduodenal tube comprising steps of:
inserting an insertion tube (31) of an endoscope (3) from a second port (22) of a three-way connector (2) then into the proximal end of a placing tube (1) via a first port (21) of the three-way connector (2), through the bending section (311) of the insertion tube (31), and out of the distal end of the placing tube (1);
entering the placing tube (1) into a natural tract of a human body;
pumping air or fluid into the natural tract of the human body via the third port (23) of the three-way connector (2) in order to inflate a cavity of the human body, if needed; and
an image-capturing device (32) of the endoscope (3) capturing images, which are shown on the display unit (4) instantaneously, the placing tube (1) being guided into a target position by viewing the real-time images shown on the display unit (4).

17. A method for aiding placement of a nasogastric tube or a nasoduodenal tube according to claim 16, wherein in the step of inserting an insertion tube (31) of an endoscope (3) from the second port (22) of the three-way connector (2) then into a proximal end of a placing tube (1) via a first port (21) of the three-way connector (2), the endoscope (3) is combined with a cover (33) through a hole structure (331) of the cover (33) then fixed to the second port (22) of the three-way connector (2) so as to let the cover (33) cover the second port (22).

18. A method for aiding placement of a nasogastric tube or a nasoduodenal tube according to claim 16, wherein in the step of an image-capturing device (32) of the endoscope (3) capturing images, which are shown on the display unit (4) instantaneously, the placing tube (1) being guided to a target position by viewing the images and using a direction controller (5) to control the axial direction of the distal end of the endoscope (3) to let the placing tube (1) arrive in the target position.

19. A method for aiding placement of a nasogastric tube or a nasoduodenal tube according to claim 16, wherein in the step of entering the placing tube (1) into a natural tract of a human body with a light source emitting a light into the natural tract of the human body.

20. A method for aiding placement of a nasogastric tube or a nasoduodenal tube according to claim 19, wherein the step of the light source emitting the light into the natural tract of the human body, the images captured by the image-capturing device (32) contain information of internal organs/tissues reflected by the light, the images with information of internal organs/tissues being displayed on the display unit (4).
